(19) United States
(12) Patent Application Publication
(10) Pub. No.: US 2011/0152679 A1
(43) Pub. Date: Jun. 23, 2011

(54) SYSTEMS AND METHODS FOR GENERATING COLORED PERSISTENCE IMAGES IN NUCLEAR MEDICINE IMAGING

(76) Inventor: David Morag, Tirat Carmel (IL)

(21) Appl. No.: 12/640,761

(22) Filed: Dec. 17, 2009

(51) Int. Cl. A61B 6/00

(52) U.S. Cl. 600/431; 600/436

(57) ABSTRACT

Systems and methods for generating persistence images in nuclear medicine (NM) imaging are provided. One method includes acquiring a nuclear emission image of a patient injected with a radiopharmaceutical in a persistence data acquisition mode. The method further includes determining an assigned display color corresponding to NM persistence image information including detected nuclear activity from the radiopharmaceutical for each of a plurality of event count values. The method also includes color mapping the acquired NM persistence image information using the assigned display colors and generating with a processor a color NM persistence image based on the color mapping. The method additionally includes displaying the generated color NM persistence image.

### BACKGROUND OF THE INVENTION

The subject matter disclosed herein relates generally to nuclear medicine (NM) imaging systems, and more particularly to systems and methods for generating images of a patient in NM imaging systems, particularly for patient positioning to perform scans with NM imaging systems.

NM imaging systems, for example, Single Photon Emission Computed Tomography (SPECT) and Positron Emission Tomography (PET) imaging systems, use one or more image detectors to acquire imaging data, such as gamma ray or photon imaging data. The image detectors may be gamma cameras that acquire two-dimensional views of three-dimensional distributions of emitted radionuclides (from an injected radioisotope) from a patient being imaged.

In order to acquire NM imaging information for a region of interest (ROI), the ROI, such as a heart of a patient, must be positioned within a field-of-view (FOV) of the gamma camera. In some NM imaging studies, the ROI is positioned during a persistence imaging phase. During this persistence phase of imaging, the gray level noisy images, for example, in cardiac SPECT imaging, makes the identification of the heart organ very difficult, thereby resulting in difficulty positioning the patient such that the heart is in the middle of the view and within the FOV of the gamma camera. Thus, a high level of experience is needed by a technician in order to locate the heart in the FOV of the gamma camera during the persistence imaging phase. In some instances, even an experienced technician has difficulty locating and properly positioning the ROI within the FOV of the gamma camera. As a result of the difficulty in positioning the ROI in the FOV of the gamma camera during the persistence phase, patient rescanning and sometimes even misdiagnosis can result.

### BRIEF DESCRIPTION OF THE INVENTION

In accordance with various embodiments, a method for generating nuclear medicine (NM) persistence images is provided. The method includes acquiring a nuclear emission image of a patient injected with a radiopharmaceutical in a persistence data acquisition mode. The method further includes determining an assigned display color corresponding to NM persistence image information including detected nuclear activity from the radiopharmaceutical for each of a plurality of event count values. The method also includes color mapping the acquired NM persistence image information using the assigned display colors and generating with a processor a color NM persistence image based on the color mapping. The method additionally includes displaying the generated color NM persistence image.

In accordance with other embodiments, a user interface for nuclear medicine (NM) persistence phase imaging is provided. The user interface includes a settings portion including at least one selectable element that is selectable by a user interface selection device to initiate generation of a color persistence image based on color mapping using NM event counts in a persistence image phase. The user interface further includes an image display portion displaying the color persistence image.

In accordance with yet other embodiments, a nuclear medicine (NM) imaging system is provided that includes at least one imaging detector configured to acquire image information in a persistence imaging phase. The acquired image information includes event count information. The NM imaging system further includes a color mapping module configured to map display colors based on the event count information and a color persistence image generating module configured to generate color persistence images based on the mapping of the display colors.

### BRIEF DESCRIPTION OF THE DRAWINGS

FIG. 1 is a flowchart of a method for performing a nuclear medicine (NM) persistence scan in accordance with various embodiments.

FIG. 2 is a flowchart of a color map setup method in accordance with various embodiments.

FIG. 3 is a color mapping table formed in accordance with various embodiments.

FIG. 4 is a diagram illustrating an NM imaging system in which various embodiments may be implemented.

FIG. 5 is a diagram of a detector of the NM imaging system of FIG. 4.

FIG. 6 is a user interface provided in accordance with various embodiments displaying color persistence images.

FIG. 7 is the user interface of FIG. 6 showing a different display of color persistence images.

FIG. 8 is the user interface of FIG. 6 showing grayscale persistence images.

FIG. 9 is a flowchart illustrating an NM imaging workflow in accordance with various embodiments.

### DETAILED DESCRIPTION OF THE INVENTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrase “reconstructing an image” is not intended to exclude embodiments in which data representing an image is generated, but a viewable image is not. Therefore, as used herein the term “image” broadly refers to both viewable images and data representing a viewable image. However, many embodiments generate, or are configured to generate, at least one viewable image.

Various embodiments provide systems and methods for generating images for display during a persistence phase of nuclear medicine (NM) imaging. During the persistence phase of NM imaging, color images are generated and displayed to a user, which may be used, for example, to position a region of interest (ROI) of a patient within a field-of-view (FOV) of a gamma camera. For example, a hot/cold colored persistence image may be generated showing a currently imaged region of a patient.

It should be noted that the persistence phase image is generated during a mode of an NM imaging wherein an image is being generated by the emission of an administered radiopharmaceutical, but the imaging system is not acquiring data. For example, the persistence phase refers to an NM imaging phase or mode wherein an image is generated from emission data acquired currently or over a previous period, such as one or two seconds, thereby generating a lower resolution image. In the persistence mode, a larger amount of statistical emission data is generally not acquired and processed, such as in an NM image study. Thus, in accordance with various embodiments, during the persistence imaging phase, diagnostically relevant data is not being acquired.

A technical effect of at least some embodiments is the enablement of easier viewing of persistence images and identification of an ROI of a patient, which may be used for positioning the ROI within a FOV of a gamma camera. NM persistence imaging practiced in accordance with the various embodiments may provide faster scan throughput and reduced patient rescans.

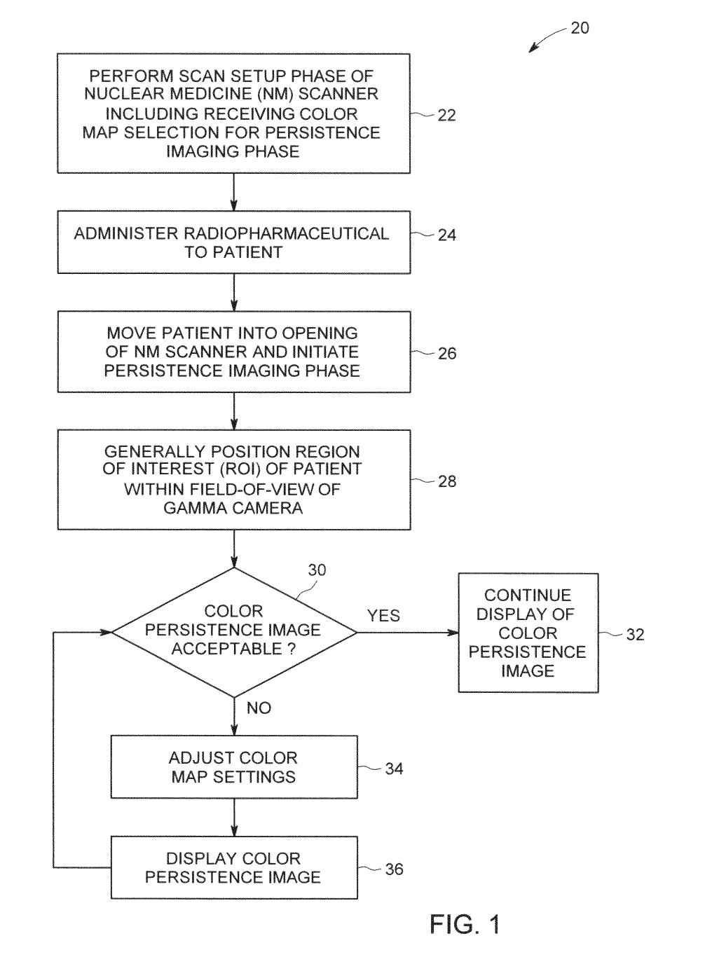

Specifically, various embodiments provide a method 20 as illustrated in FIG. 1 for performing an NM persistence scan, which may be used, for example, as part of a dynamic NM study or other diagnostic study. An NM study generally refers to acquiring NM data using one or more NM scans. The gamma cameras used for acquiring the images may be different types of gamma detectors and have different types of collimators that create different size FOVs.

The method 20 includes performing a scan setup phase of an NM scanner at 22. The scan setup phase in accordance with various embodiments includes receiving a color map selection for a persistence imaging phase. The received color map selection may be a default setting, may include user defined settings or changes to the default settings, or a combination thereof. The setup phase also may include determining or defining other operating or scan parameters, for example, setting up scan protocol settings, etc.

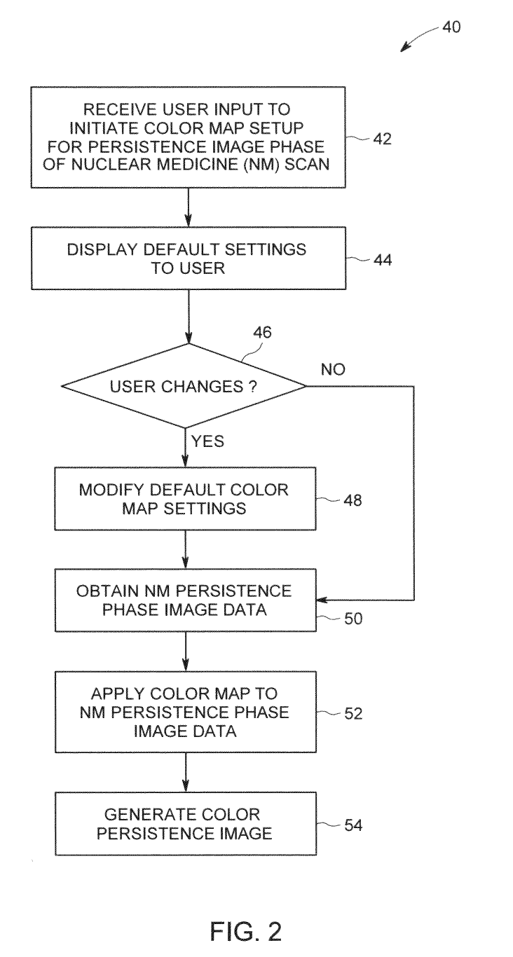

In various embodiments, a color map setup method 40 as shown in FIG. 2 may be performed as part of the scan setup phase. The method 40 includes receiving a user input at 42 to initiate the color map setup for a persistence image phase of an NM scan. The user input may be received as part of the scan setup at 22 in the method 20 of FIG. 1. It should be noted that if default settings are used or a user does not request any change to the default settings, the method 40 initially may not be performed.

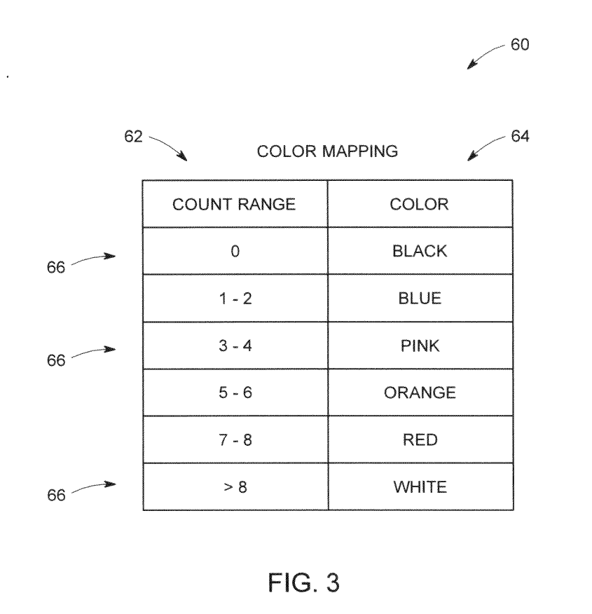

The method 40, once initiated, includes displaying default settings at 44, which includes default color map settings, which may be displayed to a user. For example, as shown in FIG. 3, color mapping settings, which may be provided in a color mapping table 60 are displayed to a user. The color mapping table 60 generally assigns a color to nuclear activity, such as to an NM event count value (e.g., photon count value), which may be used to define the color for each pixel displayed in a persistence image as described in more detail herein. The color mapping table 60 include a count range column 62 and a color column 64. The count range column 62 defines a count range for a corresponding color in the color column 62. Accordingly, each row 66 of the color mapping table 60 defines a color to be displayed for a particular event count value. The illustrated color mapping table 60 illustrates a hot/cold color mapping scheme that may be used to generate colored persistence images. Thus, for example as shown in the table 60, which shows illustrative values, if a particular image pixel corresponds to an event count of 1, the pixel will be colored and displayed blue in the persistence image and if the image pixel corresponds to an event count of 5, the pixel will be colored and displayed orange in the persistence image.

The color map settings may be stored, for example, as a predefined color map file, with color mapping values read into memory to map persistence image pixel nuclear activity into a predetermined pixel color. In various embodiments, higher image pixel activity is mapped into colors more easily distinguishable from the background, for example, brighter or hotter colors. In some embodiments, the color mapping values may be defined by a color map long value that is transformed into Red Green Blue (R.G.B.) color values, for example, using the following formula:

R: Value % 256

G: (Value/256) % 256

B: (Value/256/256) % 256

These R.G.B. values thereby define a color value for each pixel based upon on associated activity, such as an event count for each pixel. Thus, for example, in order to map pixel activity to a specific color, the various embodiments may use or implement a color map file, where different colors or shades thereof are defined by color values ranging from 0 to 256. The numbers represent a desired color per pixel activity. Each of the numbers is defined as followed:

$${2}^{8} \qquad {2}^{8} \qquad {2}^{8}$$

$${2}^{8} = 256$$

Accordingly, the R.G.B. value may be a single number in the color map file. Thus, each 0 . . . 255 number per R.G.B. provides 256 multiples by three color options. In operation, one or more color map files are accessed and a color mapping structure is determined and stored in memory, which is used to assign a color value that replaces, for example, the gray level color.

The formula described above is used to extract the (R,G,B) values (0 . . . 255,0 . . . 255,0 . . . 255) from each integer number that is stored within the color map file, which may be provided, for example, for storage space reduction. Accordingly, varied color map files may be generated and that allow a user to select a color scheme as desired or needed. In operation, in some embodiments, the values are transformed to binary, with the result being a 24 bit long number. It should be noted that zeros may be added to the left side (most significant bits) of the number to complete the number if the number is not 24 bits in length. The number is split to three 8 bit long parts: r—8 most significant bits; b—the 8 least significant; and g—central 8 bits. The 8 bits numbers (R,G,B) are then used for generating the different colored pixels. For example, the display may use 8 bits×3 for Red, Green and Blue values in order to define a specific color with the display (e.g., display card) provided with the binary values as described in more detail above.

It should be noted that the colors may correspond to a particular event count value as described in more detail herein. Accordingly, various embodiments also may provide a conversion from NM values (e.g., 5 counts) to a map value, for example, scaling by 10⁶=>5,000,000. In some embodiments, the NM value mapping may be performed using normalization into color map values based upon the maximum and minimum values in the specific display or scene. It also should be noted that in some embodiments the color selected is the color associated with a map value equal (or just less than) the scaled NM value.

Thus, as described in more detail herein, and for example when performing a cardiac NM study, the color map allows for the identification of a patient's heart using the heart high nuclear activity pixels that are brighter and isolated from the background noisy pixels.

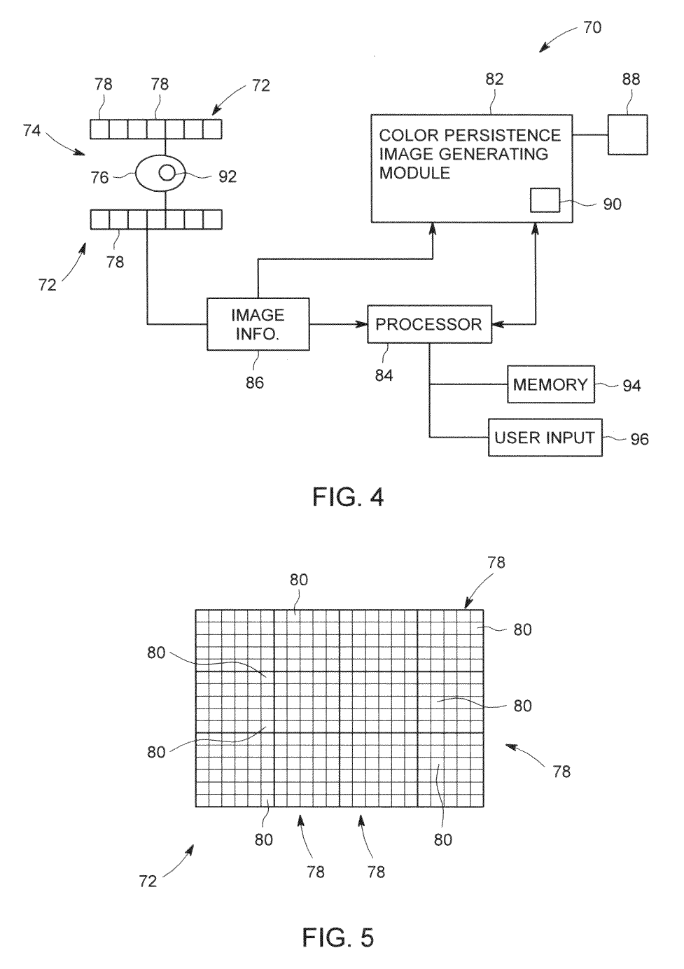

Each pixel generally corresponds to a pixel of an NM camera in an NM imaging system 70, for example, as shown in FIG. 4. NM imaging, including the persistence imaging phase may be performed using the imaging system 70. The imaging system 70 includes one or more detectors, such as a pair of detectors 72 having a central opening 74 therethrough. The opening 74 is configured to receive an object therein, such as a patient 76. The detectors 72 are pixelated detectors configured to operate in an event counting mode. The pixelated detectors 72 may be configured to acquire SPECT image data, for example, in a persistence imaging phase. The detectors 72 may be formed from different materials, particularly semiconductor materials, such as cadmium zinc telluride (CdZnTe), often referred to as CZT, cadmium telluride (CdTe), and silicon (Si), among others. In some embodiments, a plurality of detector modules 78 are provided, each having a plurality of pixels 80 a shown in FIG. 5 and forming a detector 72. Alternatively, the detector 72 may be made of a scintillation crystal such as Sodium Iodide (Nap and an array of Photo-Multiplier Tubes (PMTs) as known in the art. In general, the detectors 72 are fitted with collimators.

The detectors 72 may be provided in different configurations, for example, in an “L” mode configuration, but may be moved and positioned in other configurations such as an “H” mode configuration. Additionally, a gantry (not shown) supporting the detectors 72 may be configured in different shapes, for example, as a “C”, “H” or “L”. It should be noted that more or less detectors 72 may be provided.

The imaging system 70 also includes a color persistence image generating module 82 that implements the various embodiments, including the method 20 (shown in FIG. 1) and the method 40 (shown in FIG. 2). The color persistence image generating module 82 may be implemented in connection with or on a processor 84 (e.g., workstation) that is coupled to the imaging system 70. Optionally, the color persistence image generating module 82 may be implemented as a module or device that is coupled to or installed in the processor 84. During operation, the output from the detectors 72, which may include image information 86, such as NM persistence image information, is transmitted to the color persistence image generating module 82. However, other image information or data may be output from the detectors 72, such as NM image study data that is statistically correlated to generate diagnostic NM images.

The color persistence image generating module 82 is configured to receive the image information 86, and in particular, activity information such as event counts from a current detection, for example, counts currently acquired or acquired over a few seconds (e.g., one, two or three seconds), and generate a color persistence image based on the image information 86 to form a color persistence image 88. More specifically, in the exemplary embodiment, the color persistence image generating module 82 uses event counts and color mapping as described in more detail herein to generate colored pixels for display as a persistence image, which mapping may be provided by a color mapping module 90. The color persistence image generating module 82 and/or the color mapping module 90 may be implemented as a set of instructions or an algorithm installed on any computer that is coupled to or configured to receive the image information 86, for example, a workstation coupled to and controlling the operation of the imaging system 70.

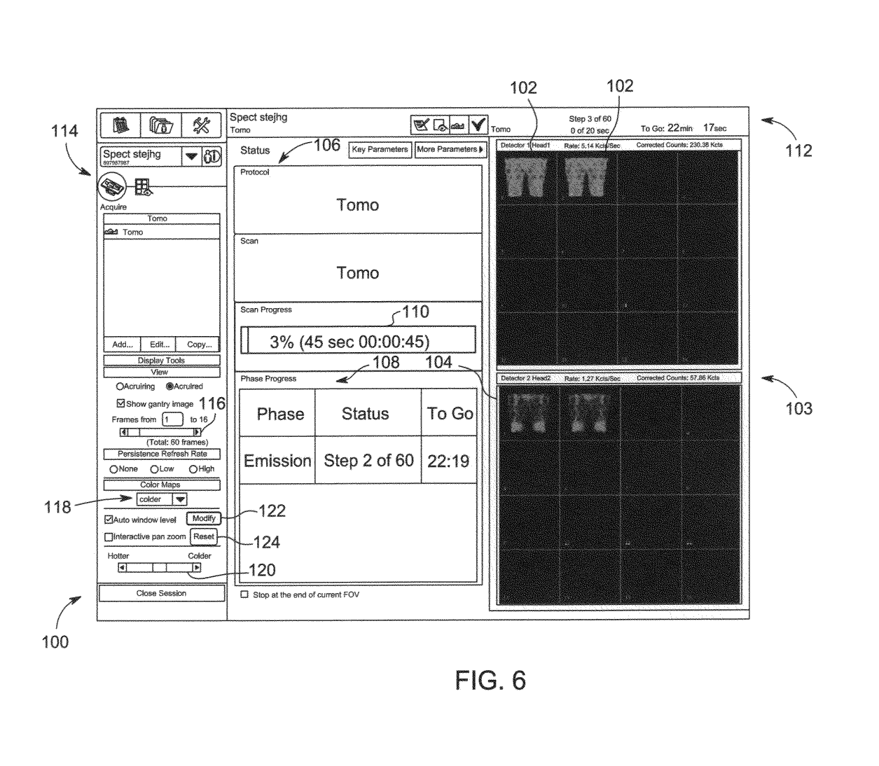

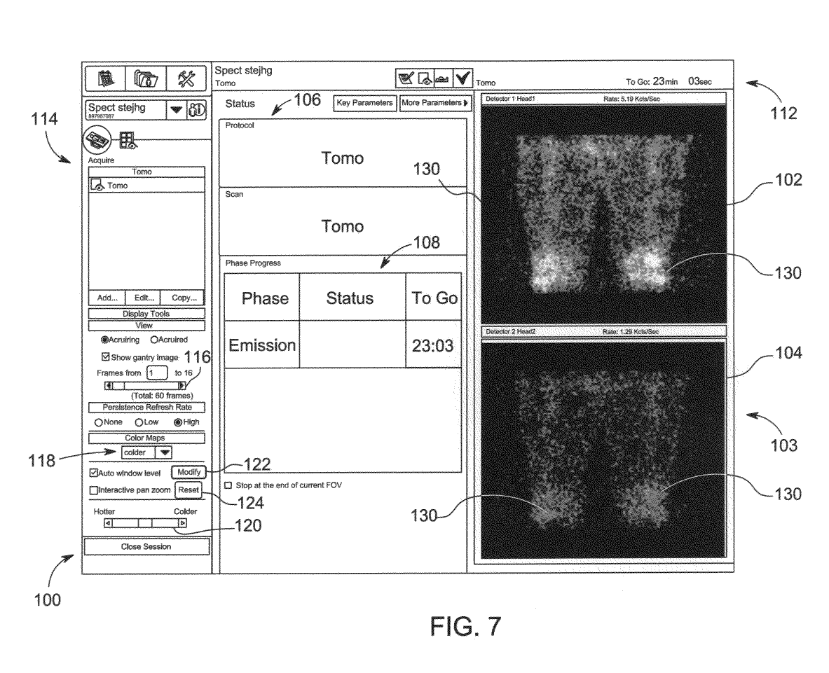

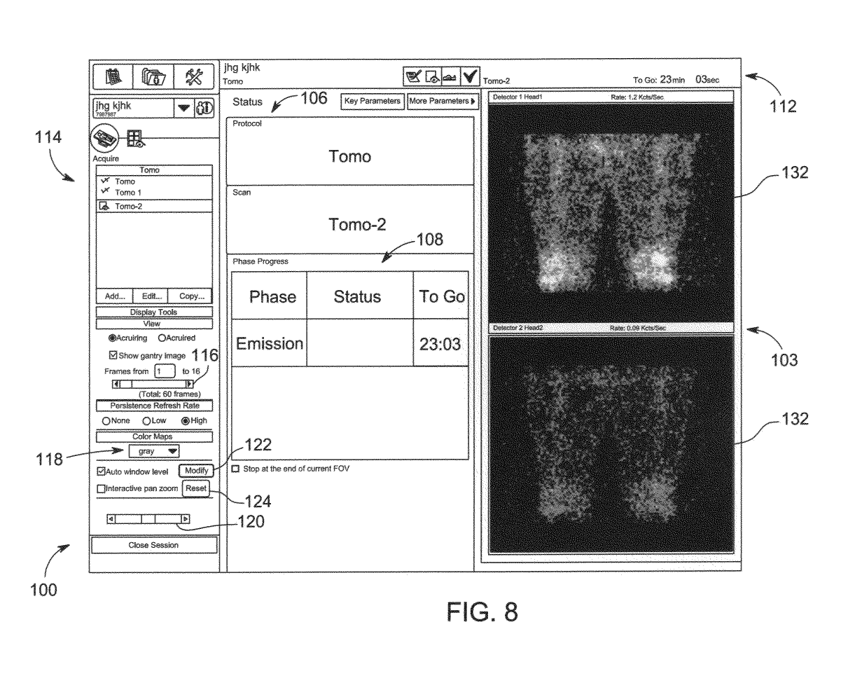

Thus, for example, event count information, such as photon count information from a region of interest 92 (e.g., heart, lung, knee, etc.) of the patient 76 is obtained from the modules 78 of the detectors 72. As shown in FIGS. 6 and 7, and as described in more detail herein, the image information 86, which may be SPECT persistence image information for a leg of a patient, is displayed as a colored, such as hot/cold colored image map, which may be used to position a knee of a patient in the FOV of the detectors 52 for a subsequent diagnostic NM study. It should be noted that the various embodiments may also display gray scale persistence images as shown in FIG. 8 and described in more detail herein, which images are pre-color mapped images. It should be noted that the grayscale persistence images and color persistence images may be displayed concurrently.

It also should be noted that the raw data, such as the image information 86, or color mapped persistence data may be stored for a short term (e.g., during processing) or for a long term (e.g., for later offline retrieval) in a memory 94. The memory 94 may be any type of data storage device, which may also store databases of information. The memory 94 may be separate from or form part of the processor 84. A user input 96, which may include a user interface selection device, such as a computer mouse, trackball and/or keyboard is also provided to receive a user input, such as a change to the color mapping as described in more detail herein.

Referring again to the method 40 of FIG. 2, after the default settings for color mapping are displayed to a user, a determination is made at 46 as to whether there are any changes. For example, a user may change the count range corresponding to a particular color or change the color to which a particular count range is to be mapped. The count range may be increased, decreased, narrowed or widened. The count ranges may be changed individually or as a group, for example, adding to or subtracting from each of the count ranges as a group to make the overall color persistence image hotter or cooler. The user may also save the changed mapping settings as a custom set of preferences, for example, for use in future types of similar NM studies, such as NM studies of a particular body part.

If a determination is made at 46 that user changes have been made, then at 48 the default color map settings are modified for the current NM scan or study. Thereafter, or if a determination is made at 46 that there are no user changes, NM persistence phase image data is obtained at 50, which is acquired after administration of a radiopharmaceutical as described in more detail below in connection with the method 20 of FIG. 1. For example, current emission photon count information or photon count information over a shorter period of time is obtained at 50. In various embodiments, the persistence phase image data is any data that is not NM diagnostic data acquired for generating diagnostically relevant images. The NM persistence phase image data shows a current patient image representation based on a number of counts that are not necessarily stored for an NM study.

Thereafter, at 52 the color map is applied to the NM persistence phase image data, for example, on a pixel by pixel basis based on the color mapping count ranges. Accordingly, for photon counts detected by each pixel of a detector module of an image detector, a colored persistence image pixel is generated at 54 to form a color persistence image.

Referring again to the method 20 of FIG. 1, after the color mapping setup (as well as other scan setup) is performed, a radiopharmaceutical is administered to a patient at 24. It should be noted that different types of radiopharmaceuticals may be used, such as based on the type of NM study to be performed. For example, the radiopharmaceuticals that may be used in accordance with various embodiments (administered intravenously) include: Technetium-99m (technetium-99m), Iodine-123 and 131, Thallium-201, Gallium-67, Fluorine-18 Fluorodeoxyglucose and Indium-111 Labeled Leukocytes. Other examples of the radiopharmaceuticals that may be used in accordance with various embodiments (administered in gaseous form) include: Xenon-133, Krypton-81m, Technetium-99m Technegas and Technetium-99m DTPA.

The administration of the radiopharmaceutical may be performed in any suitable manner, which may be based on the type of radiopharmaceutical selected (e.g., intravenous versus gaseous). In general, the radiopharmaceutical dose is administered internally (e.g. intravenous or orally). The radiopharmaceutical then uptakes into the patient's body, and in particular the ROI of the patient. The radiopharmaceutical is produced to localize in an organ or body structure of interest. The radiopharmaceuticals may be formed from radionuclides that are combined with other chemical compounds or pharmaceuticals. The radiopharmaceutical, once administered to the patient, thus, localizes to specific organs or cellular receptors.

After administration of the radiopharmaceutical, the patient is moved into an opening of the NM scanner, which may be performed automatically, based on user input, or a combination thereof, and a persistence imaging phase is initiated at 26 such the NM scanner begins acquiring persistence images. The ROI of the patient is thereafter generally positioned within the FOV of the gamma camera at 28, such as based on a generally known location of a particular region or organ within a patient. A determination is then made at 30 whether the color persistence image is acceptable. For example, a user may determine whether the color mapping allows for identification of an ROI of patient, such as to distinguish the ROI (identified as a “hot” portion of the image based on pixel color) from the background or other objects within the patient.

If the color persistence image is acceptable, the display of the color persistence image is continued at 32. The display of the color persistence image allows, for example, for an ROI of a patient to be positioned within the FOV of a gamma camera for an NM study. If the color persistence image is not acceptable at 30, the color map settings may be adjusted at 34. For example, the count ranges for the color mapping may be modified as described in more detail herein, such as in connection with the method 40 of FIG. 2. Thereafter, a color persistence image with the adjusted color mapping is displayed at 36. A determination is then made again at 30 as to whether the color persistence image is acceptable.

Thus, as shown in the user interfaces of FIGS. 6 and 7, one or more color persistence images may be displayed. In particular, as shown in the user interface 100 of FIG. 6, a plurality of color persistence images 102 and 104, which correspond to images acquired by different image modules (each image represents a persistence image generated from one or more modules) in different detectors or detector heads may be displayed in an image display portion 103. The user interface 100 also includes a status portion 106 and a phase progress portion 108. The status portion 106 indicates the type of NM scan being performed and a current progress of the scan, for example, using a status indicator 110. The phase progress portion 108 indicates the status of the current phase, such as an emission phase of an NM study. The phase progress information may be displayed in other portions of the user interface 100, for example, at 112.

A settings portion 114 is also displayed, which is illustrated as a setup and settings panel. The settings portion 114 allows a user to adjust the current view, such as by varying the number of frames to be displayed using a slider bar 116. Additionally, the settings for the color persistence images 102 and 104 being displayed may be adjusted, such as a persistence refresh rate, which defines the time period for updating the color persistence images 102 and 104, such as every one, two or three seconds. Additionally, the color mapping settings may be adjusted or modified as described in more detail herein. For example, a contrast ratio for the color persistence images 102 and 104 may be set using a color setting field 118, which may define a color temperature setting or a grayscale setting (illustrated as a colder image setting). For example, the field 118 may be used to determine whether colder colors (such as more greens) or hotter colors (such as more reds) are to be used to generate the color persistence images 102 and 104 and/or indicate higher nuclear activity as described herein. A user may also adjust the color settings of the color persistence images 102 and 104, such as the color temperature range of the mapping used to generate the color persistence images 102 and 104 using a slider bar 120.

Additionally, the color mapping may be selectively modified, such as by individually changing the nuclear activity or photon count ranges in the color mapping (for example, as shown in FIG. 3) or the colors by selecting a Modify selectable element 122. Selection of the Modify selectable element 122 may cause the user interface 100 to display table, such as the color mapping table 60 of FIG. 3 to allow more selective modification of the color mapping. It should be noted that additional count ranges or count numbers as well as additional colors may be added to the color mapping table 60. The default settings (default color mapping settings) may be reset using the Reset selectable element 124.

It should be noted that the slider bars and other selectable elements may be displayed as virtual elements of the user interface 100, which are selectable or operable, using a user interface selection device, such as a computer mouse, trackball and/or keyboard that receives a user input. It also should be noted that the user interface 100 may be displayed on a display of an NM imaging scanner or workstation.

FIG. 7 shows the user interface 100 wherein single color persistence images 102 and 104 are displayed in an enlarged format. The color persistence images 102 and 104 are NM persistence images of a leg and pelvis region of a patient with a radiopharmaceutical administered to a patient, which uptakes into the knees of the patient, identified as the colored hot spots 130 (e.g., more red/orange than blue regions) of the color persistence images 102 and 104. As can be seen, the various embodiments provide color mapping for a patient image viewer that includes a color dimension illustrated as a hot/cold colored image map. The “hot” region, which indicates the ROI based on higher nuclear activity from radiopharmaceutical uptake, is distinguishable from the colder region or background by the color mapping. Accordingly, regions having higher nuclear activity or photon count information are colored hotter than regions with lower photon count information. It should be noted that the user interface 100 also may display gray scale persistence images 132 as illustrated in FIG. 8, wherein gray scale mapping is performed.

Thus, in accordance with various embodiments, NM persistence images are color mapped, which includes assigning a color or value to each pixel that is proportional to nuclear activity, such as the number of event counts (e.g., photon counts). For example, each pixel color is defined by the corresponding event counts for the previous one, two or three seconds. In general, in the persistence image phase, images are generated, but NM study information is not acquired, for example, statistically relevant event count information is not acquired and stored. The color mapping may be performed continuously or periodically as the counts for the persistence images are updated.

In some embodiments, a full scale of colors is used for the color mapping. The colors may be automatically determined or modified as described herein. For example, in various embodiments, the number of counts corresponding to a white color (the brightest color), which is the highest count is defined, with the other colors down to black, which is the lowest count, linearly mapped therefrom. The counts may be scaled up or down, for example, based on an amount of count activity. The scaling may be performed automatically, manually, associated with a specific imaging protocol etc. For example, in automatic scaling, the image is periodically searched and the maximal value is determined. The value for maximal brightness is then set as the maximal value determined. Imaging protocols are often used for ease of setting up the camera or detector for a specific imaging type. Each protocol defines several imaging parameters, for example energy windows, acquisition time, etc. In some embodiments, the color map and/or scaling for color representation is stored and is associated with one, several or all of a number of clinical protocols. The color map also may be reversed, displaying higher values as darker colors, and lower values as brighter colors. In some embodiments, a user may select a “color negative” display to reverse the color representation.

Variations and modifications are contemplated. For example, persistence image information may be tracked for a longer period of time, such as ten seconds in order to adjust a contrast ratio for the color mapping, which may be used when the ROI is in a region where the count value does not vary much, such as in the torso of a patient (for a cardiac image) versus in the legs of patient (for a knee image). When the image is tracked for a longer period of time or an extended time period, such that more counts are obtained, a scaling factor or value may be subtracted from all the counts (before color mapping) to reduce the background noise. However, it should be noted that the scaling factor or value is selected such that all scaled values are positive (or where negative values are discarded).

As another example of a modification to the various embodiments, a gain factor or value may be applied such that all of the counts are multiplied by the gain factor or value (before color mapping) to increase a brightness of the image. Similarly, a reduction factor or value may be applied by division of the counts. As still another example of a modification to the various embodiments, a dynamic scale may be used at each point in time to obtain good color characteristics to distinguish the ROI in the persistence images.

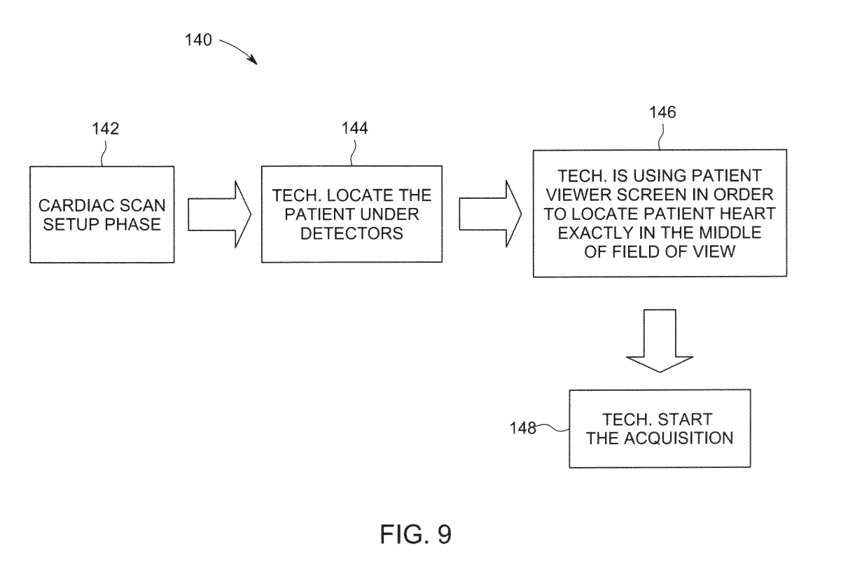

Thus, as shown in FIG. 9, various embodiments provide color persistence images as part of an NM imaging workflow 140, which is illustrated as a cardiac NM study. In particular, at 142 a cardiac scan setup phase is initiated, which may include setting up the color mapping of the various embodiments. A technician then locates a patient under the detectors of an NM scanner at 144 and initiates a persistence imaging phase. Thereafter, the technician uses a patient viewer screen at 146, which may include one or more of the user interfaces of the various embodiments, to locate a patient heart in the middle of the FOV of the detectors using one or more color persistence images. The color mapping also may be modified as described in more detail herein. Once the heart is located within the middle of the FOV of the detectors, an acquisition phase is initiated at 148, which includes acquiring and storing nuclear activity or event count information for a diagnostic NM study.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for providing nuclear medicine (NM) persistence images, the method comprising:
acquiring a nuclear emission image of a patient injected with a radiopharmaceutical in a persistence data acquisition mode;
determining an assigned display color corresponding to NM persistence image information including detected nuclear activity from the radiopharmaceutical for each of a plurality of event count values;
color mapping the acquired NM persistence image information using the assigned display colors;
generating with a processor a color NM persistence image based on the color mapping; and
displaying the generated color NM persistence image.

2. A method in accordance with claim 1 further comprising assigning a display color to at least one range of event count values.

3. A method in accordance with claim 1 wherein the mapping is performed on a pixel by pixel basis of a detector acquiring the NM persistence image information.

4. A method in accordance with claim 1 wherein the NM persistence image information is acquired over a time period of not more than two seconds.

5. A method in accordance with claim 1 further comprising changing the count values for assigning the display colors based on a user input.

6. A method in accordance with claim 1 further comprising changing the display colors to assign to the count values based on a user input.

7. A method in accordance with claim 1 further comprising scaling the count values prior to assigning the display colors.

8. A method in accordance with claim 1 wherein the count values are determined based on a linear scaling from a highest count value.

9. A method in accordance with claim 1 further comprising adjusting a color temperature for the assigned display colors.

10. A method in accordance with claim 1 wherein the NM color persistence image comprises a hot/cold colored image map.

11. A method in accordance with claim 1 further comprising adjusting a contrast ratio of the mapping by subtracting a scaling factor from each of the count values prior to the color mapping, wherein an extended time period is used for determining the count values.

12. A method in accordance with claim 1 wherein the display colors comprise a full scale of colors and further comprising automatically determining the color scale.

13. A method in accordance with claim 1 further comprising continuously updating the color NM persistence image.

14. A method in accordance with claim 1 wherein the nuclear emission image is a two-dimensional nuclear emission image acquired by a nuclear camera of an NM imaging system, and further comprising positioning a region of interest of a patient within a field of view of the nuclear camera using the color NM persistence image.

15. A user interface for nuclear medicine (NM) persistence phase imaging, the user interface comprising:
a settings portion including at least one selectable element that is selectable by a user interface selection device to initiate generation of a color persistence image based on color mapping using NM event counts in a persistence image phase; and
an image display portion displaying the color persistence image.

16. A user interface in accordance with claim 15 wherein the settings portion comprises at least one other selectable element that is selectable by a user interface selection device to modify a color mapping used to generate the color persistence image.

17. A user interface in accordance with claim 15 wherein the color persistence image comprises a hot/cold colored image map.

18. A user interface in accordance with claim 15 wherein the at least one other selectable element comprises a slider bar configured to adjust a range of NM event counts for the color mapping to generate the persistence image.

19. A nuclear medicine (NM) imaging system comprising:
at least one imaging detector configured to acquire image information in a persistence imaging phase, the acquired image information including event count information;
a color mapping module configured to map display colors based on the event count information; and
a color persistence image generating module configured to generate color persistence images based on the mapping of the display colors.

20. An NM imaging system in accordance with claim 19 further comprising a user input configured to receive a user input to change the color mapping.
